(19) United States
(12) Patent Application Publication
(10) Pub. No.: US 2014/0221759 A1
(43) Pub. Date: Aug. 7, 2014

(54) PUPIL EXPANSION APPARATUS

(71) Applicant: Impex, Inc., Brooklyn, NY (US)

(72) Inventors: Richard Jonathan Mackool, Astoria, NY (US); Aaron Beller, Bellmore, NY (US); Joseph Beller, Brooklyn, NY (US)

(73) Assignee: Impex, Inc., Brooklyn, NY (US)

(21) Appl. No.: 14/174,288

(22) Filed: Feb. 6, 2014

Related U.S. Application Data
(60) Provisional application No. 61/761,457, filed on Feb. 6, 2013.

(51) Int. Cl. A61B 17/02

(52) U.S. Cl. CPC A61B 17/0231
USPC 600/209; 600/206

(57) ABSTRACT

A pupil expansion apparatus is placed in the eye during an ophthalmic surgical procedure. In one embodiment, the pupil expansion apparatus is a square frame structure that comprises four straight beams which cross each other at their ends to form a cross beam arrangement. However, the expansion apparatus is not limited to being a square frame structure, and may have a circular, or any multi-sided frame structure. The crossed ends of the straight beams are connected by one or more curved beams, which also form iris supporting portions. The iris supporting portion is shaped in an open, curved form so as to be perpendicular to the plane formed by the frame. It receives the rim of the iris tissue when the pupil expansion apparatus is in its extended position and gently presses the iris to the periphery.

### CROSS-REFERENCE TO RELATED PATENT APPLICATIONS

This application claims priority to U.S. Provisional Application Ser. No. 61/761,457 filed on Feb. 6, 2013, which is incorporated by reference herein in its entirety.

### FIELD OF THE INVENTION

This invention relates to a pupil expansion apparatus that is placed in the eye during an ophthalmic surgical procedure.

### BACKGROUND OF THE INVENTION

When an ophthalmic surgical procedure is performed, usually one or more incisions are made in the corner of the eye to allow the introduction of surgical instruments. This type of procedure requires dilation of the pupil, i.e., the hole at the center of the iris, so as to provide the surgeon with a wide opening through which the surgeon can view and work on the posterior portions of the eye. Dilation involves enlarging the pupil by causing the iris tissue to retract.

Traditionally, a chemical dilation method using a mydriatic solution has been employed for this purpose. However, since this method does not provide accurate control over the expansion of the pupil, mechanical dilation is now more commonly used in clinical settings.

For example, U.S. Pat. No. 4,257,130 issued to Bayers discloses an intraocular lens that is inserted within the posterior chamber of the eye. i.e., the region behind the iris. The lens mechanism of Bayers includes a pair of appendages, each of which has one part that passes through the pupil and another part that extends along side of the iris. This apparatus is rather simple. However, it can be traumatic to the eye since a relatively large portion of the eye may be affected or possibly damaged during a procedure with this device.

Another example is shown in U.S. Pat. No. 5,441,045 and No. 5,318,011, issued to Federman, which disclose an iris dilator comprising an expansible material that can expand from a dehydrated first size and shape to a hydrated second size and shape. When hydrated, the dilator has a shape complementary to an inside diameter of the iris and a size sufficient to dilate the iris. In addition, a mammalian iris may be dilated by inserting the at least partially dehydrated iris dilator into a position radially inwardly from the iris. In the presence of bodily or surgical fluids, the dilator hydrates and expands to engage and dilate the iris. Apparatus for deforming the dilator to facilitate insertion is also utilized. However, this method not only involves a complicated, time consuming process of dehydration/hydration, but also requires expensive materials (e.g., hydrogels) and extra apparatus (e.g., deformation apparatus).

Another method is disclosed by U.S. Pat. No. 8,323,296 of Malyugin and U.S. Publication No. 2008/0243139 of Dusek, where a ring is used to maintain a pupil in an enlarged position by pushing the iris tissue outwards during an ophthalmic procedure. The ring has three or more straight sides connected by loops that capture iris tissue. Each loop consists of upper and lower helical turns or strand portions that are at an angle to each other to form a gap facing the surrounding iris tissue. The flexibility of the ring allows the turns to deflect and apply a clamping force onto the iris tissue. The clamping force assists in maintaining the pupil in an enlarged position and also stabilizes the position of the ring relative to the eye. The flexibility also allows the ring to be collapsed for insertion into the eye.

However, in Malyugin and Dusek, because it is “clamped” by the loop, the iris tissue may be susceptible to damage depending on the skill of the surgeon who performs the procedure.

Further, the loops of Malyugin are formed on the interior of the frame of straight sides. Accordingly, when the ring is in the fully extended position, these internal loops partially block a part of the viewing area during the procedure. Access to the interior of the pupil may also be partially prevented due to these internal loops.

The present invention has been conceived in view of the above circumstances, and an object of the invention is to provide a pupil expansion apparatus that can effectively provide a less invasive procedure, a clear viewing area, and easy access to the posterior portions of the eye.

### SUMMARY OF THE INVENTION

The present invention is a pupil expansion apparatus that is placed in the eye during an ophthalmic surgical procedure. According to one embodiment of the present invention, the pupil expansion apparatus is a square frame structure that comprises four straight beams which cross each other at their ends to form a cross beam arrangement. The pupil expansion apparatus of the present invention is not limited to being a square frame structure, but it can be designed to be circular, or any multi-sided frame structure.

The crossed ends of the straight beams are connected by one or more curved beams, which also form iris supporting portions. In one embodiment of the present invention, each of cross beam connections contains five interconnected curved beams.

The iris supporting portion is shaped in an open, curved form so as to be perpendicular to the plane formed by the square frame. It receives the rim of the iris tissue when the pupil expansion apparatus is in its extended position, while gently pressing the iris to the periphery. In this manner, the iris tissue does not have to be “clamped” as required by conventional devices, thus providing a less invasive procedure.

The pupil expansion apparatus of the present invention is configured to be collapsed and placed into a small bore tube. During installation of the pupil expansion apparatus into the eye, the tube carrying the apparatus is placed in the iris of the eye through an incision. When located in the pupil, the apparatus is ejected from the tube and expands automatically to hold the tissue of the iris out of the way during an ophthalmic procedure, such as phacoemulsification.

According to another embodiment of the present invention, the pupil expansion apparatus is made of solid material, including, but not limited to, metal or plastic. In one preferred embodiment, the apparatus is made of polypropelene.

### BRIEF DESCRIPTION OF THE DRAWINGS

These and other features and advantages of the present invention will become more readily appreciated when considered in connection with the following detailed description and appended drawings, wherein like designations denote like elements in the various views, and wherein:

FIG. 1 is a perspective view of a pupil expansion apparatus in accordance with one embodiment of the present invention;

FIG. 2 is a top view of the pupil expansion apparatus of the present invention.

FIG. 2A is an enlarged top view of an iris supporting portion of the pupil expansion apparatus;

FIG. 3 is a side view of the pupil expansion apparatus;

FIG. 3A is an enlarged side view of the iris supporting portion of the pupil expansion apparatus;

FIG. 3B is an enlarged front view of the iris supporting portion of the pupil expansion apparatus;

FIG. 4 is an illustration showing the pupil expansion apparatus in the bore of a tube as it is ejected into the eye;

FIG. 5 is an illustration showing the pupil expansion apparatus in its extended position in the iris of the eye; and

FIG. 6 is a side cross-sectional view of the eye where the pupil expansion apparatus is in its extended position in contact with the iris tissue.

FIG. 7A is a perspective view of a pupil expansion apparatus in accordance with a second embodiment of the present invention.

FIG. 7B is a side view of the pupil expansion apparatus in accordance with the second embodiment.

### DETAILED DESCRIPTION OF THE INVENTION

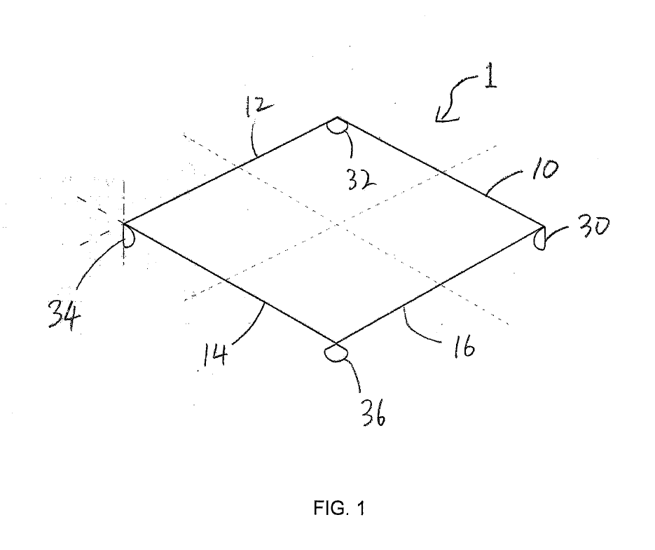

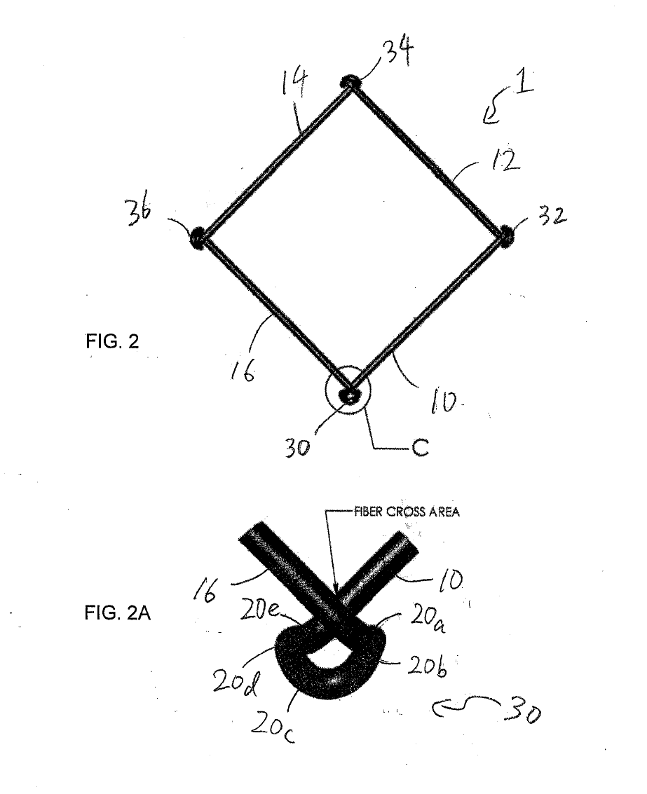

With reference to the drawings, FIG. 1 and FIG. 2 show a first embodiment of a pupil expansion apparatus according to the present invention, which is identified herein by reference number 1. The pupil expansion apparatus 1 is formed as a square frame structure comprising four straight beams 10, 12, 14, and 16 which cross each other at their ends to form a cross beam arrangement.

The pupil expansion apparatus of the present invention is not limited to being a square frame structure and it can be designed to be a rounded shape structure including circular or elliptical (not shown). Alternatively, the pupil expansion apparatus can be also designed to be any multi-sided frame structures including triangle, rectangle, hexagon, etc. (not shown)

The crossed ends of the straight beams are connected by one or more curved beams 20, 22, 24 and 26. Each of the iris supporting portions 30, 32, 34 and 36 is formed by connecting the curved beam or the straight beam at its ends. As noted in FIG. 2A, in one preferred embodiment, each of the iris supporting portions is composed of five curved beams 20a-e, 22a-e, 24a-e, or 26a-e. In one embodiment, each of straight and curved beams is connected mechanically through threads formed inside the beams. In another embodiment, each of the beams is attached using an adhesive. Also, the connection can be such as to be allow rotation or flexing of the beams with respect to each other to facilitate expansion and collapsing of the frame.

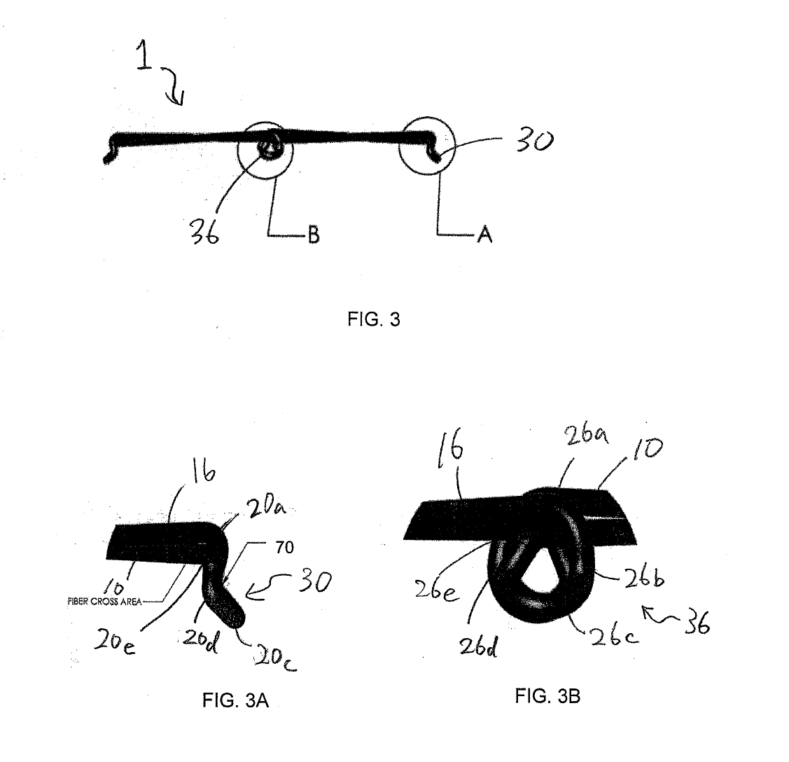

Each of the iris supporting portions 30, 32, 34 and 36 is shaped in an open, curved form so as to be perpendicular to the plane formed by the square frame of the pupil expansion apparatus 1. As shown in FIG. 3 and FIG. 3A, a receiving point 70 of the iris supporting portion receives the rim of the iris tissue when the pupil expansion apparatus 1 is in its extended position within the eye, while gently pressing the iris to the periphery of the eye.

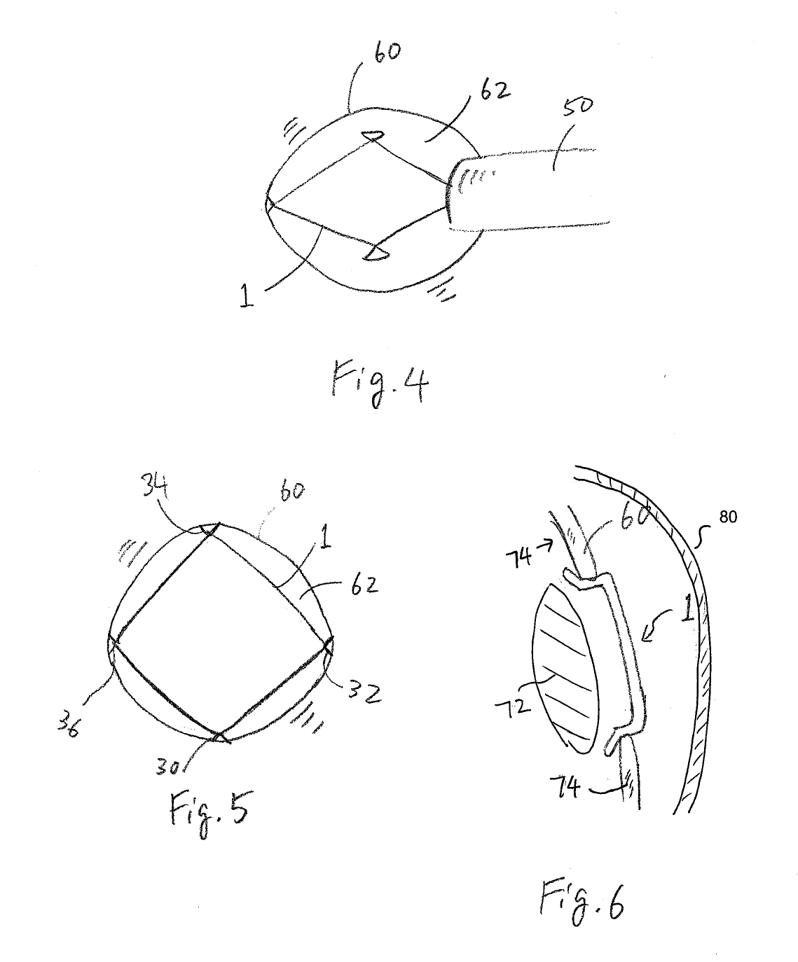

FIG. 6 illustrates the pupil expansion apparatus 1 being placed at the iris tissue 60 in its extended position. The placement can be achieved by making an incision in cornea 80 and inserting tube 50 through the incision so the apparatus 1 can be placed in the pupil (FIG. 4). Unlike conventional devices that capture the iris tissue when expanding the pupil, the pupil expansion apparatus of the present invention enlarges the pupil without capturing (i.e., vertically compressing) the iris tissue that surrounds the pupil, reducing the risk of trauma to the iris when it is installed and removed. This provides the surgeon with access to the lens 72 and the posterior portion of the eye 74.

Further, the open, curved structure of the iris supporting portion of the present invention makes it easy for the surgeon to support the iris tissue and to release it, contributing to a reduction in the operation time. On the other hand, in existing devices, the iris tissue is clamped by a loop, or it must be engaged in a groove structure, either of which would require accurate placement of the device in the eye, requiring more skill, prolonged operation time, and a more difficult removal.

As noted in FIG. 2 and FIG. 5, each of the iris supporting portions 30, 32, 34, and 36 is formed outside of the square frame, so that the pupil expansion apparatus 1 provides a viewing area that is clearer than one provided by a conventional apparatus. This “external” curved beam arrangement also provides a surgeon with easy access to the posterior portions of the eye 74.

In one preference embodiment, the angle formed at each corner of the square frame when in its extended position is 90 degrees. Preferably, the length of one side is 6.5 mm to create the pupil of 6.25-7.0 mm in diameter. Preferably, the horizontal length of the supporting portion is 1.2 mm, while the vertical length of the loop is 0.55 mm.

Because of its cross beam structure, the pupil expansion apparatus 1 can be collapsed and placed into a bore tube 50 by making the square frame into rhombic one (FIG. 4). Further, in case the apparatus is designed to be rectangular, the long sides of the straight beams can be folded towards each other to be in the collapsed position (not shown).

FIG. 4 illustrates the bore tube 50 being placed in the iris 60 of the eye through an incision. When located in the pupil 62, the apparatus 1 is ejected from the bore tube 50 and expands to hold the tissue of the iris 60 out of the way during an ophthalmic procedure. Also, thanks to this particular design, the ejection of the apparatus 1 can be performed in a relatively easy manner, providing more accurate placement of the apparatus 1.

Upon the completion of the procedure, the pupil expansion apparatus of the present invention is folded and removed by using forceps for example. Alternatively, the pupil expansion apparatus can be removed simply through the reverse action of the bore tube injection. Because the iris tissue is openly supported by the iris supporting portions 30, 32, 34 and 36, no further tool is necessary to disengage the apparatus from the iris tissue.

The pupil expansion apparatus 1 can be made of any solid material including, but not limited to, metal, plastic or plastic-coated metal. It can be made in multiple parts, but may also be made of a single strand of material. In one preferred embodiment, the apparatus 1 is made of polypropelene.

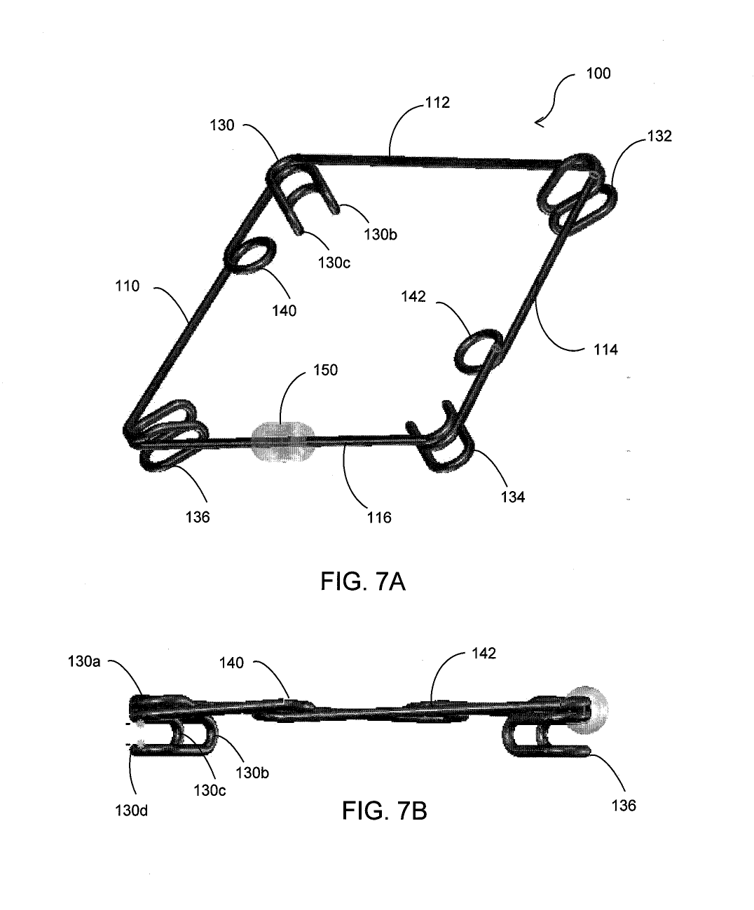

FIGS. 7A and 7B show perspective and side views of a second embodiment of a pupil expansion apparatus according to the present invention, which is identified herein by reference number 100. The second embodiment of the pupil expansion apparatus 100 is identical to the first embodiment 1, except for the cross beam overlap at the corners of the frame, as well as the design of four iris supporting portions 130, 132, 134 and 136, and the ring portions 140, 142 that are provided on straight beams 110, 114 at diagonally symmetrical positions in a plane formed by the frame structure.

In the second embodiment the ends of the straight beams 110, 112, 114, 116 have curves at their ends that overlap each other, e.g., at 130a, in the corners of the frame with one above the other. In particular, they do not have a cross beam structure. The iris supporting portions extend perpendicularly away from the plane of the frame, as in the first embodiment. However, the iris supporting portions are formed by curved beams connected to the overlapped ends of the straight beams at the frame corner, which curved beams form two generally parallel open curved shapes, e.g., 130b and 130c, directed away from the frame to capture iris tissue. The two open curved shapes are connected together at their portions perpendicularly remote from the frame corners, e.g., 130d.

In any of the embodiments, the straight and curved beams of the frame and/or the iris supporting portions may be made of a single piece of wire, tubing or the like, that is bent into the various shapes.

In one embodiment, glue 150 is attached to cover a gap formed with two ends of the straight beams 112, 116.

The ring portions 140, 142 are designed to enhance the strength and flexibility of the expansion apparatus 100 so that the surgeon can easily maneuver the apparatus during an operation. A third ring portion (not shown) may be added to further stiffen the structure of the expansion apparatus 100. The third ring portion may be provided on the straight beam 112 opposite to the beam 116 where the glue is applied.

Having described preferred embodiments of the iris expansion ring (which are intended to be illustrative and not limiting), it is noted that modifications and variations can be made by persons skilled in the art in light of the above teachings. It is therefore to be understood that changes may be made in the particular embodiments of the invention disclosed which are within the scope and spirit of the invention as outlined by the appended claims.

What is claimed is:

1. A pupil expansion apparatus comprising:
a plurality of main beams crossed at their ends to form a frame structure with corners in a plane; and
a plurality of sub beams each of which is connected at its ends to the main beam or another sub beam, wherein,
said sub beams are connected so as to form an iris supporting portion at each of the corners of the frame, and
said iris supporting portion supports iris tissue when the pupil expansion apparatus is in its extended position in the pupil.

2. The pupil expansion apparatus of claim 1, wherein the pupil expansion apparatus is collapsible.

3. The pupil expansion apparatus of claim 1, wherein the iris supporting portion is located outside of the frame structure and extends in a direction perpendicular to a plane formed by the frame structure.

4. The pupil expansion apparatus of claim 1, wherein the iris supporting portion receives the iris tissue at a receiving point.

5. The pupil expansion apparatus of claim 1 having four main beams and twenty sub beams.

6. The pupil expansion apparatus of claim 1, wherein the main beams and the sub beams are connected through threads formed inside the beams.

7. The pupil expansion apparatus of claim 1, wherein the pupil expansion apparatus can be placed into a bore tube and can be ejected from the bore tube.

8. The pupil expansion apparatus of claim 1, wherein the main and sub beams are made of plastic or metal.

9. The pupil expansion apparatus of claim 8, wherein the main and sub beams are made of polypropelene.

10. The pupil expansion apparatus of claim 1, wherein the frame structure has either a circular shape or a multi-sided shape.

11. The pupil expansion apparatus of claim 10, wherein;
the frame structure has a multi-sided shape,
the main beam has a straight shape, and
the sub beam has a curved shape.

12. The pupil expansion apparatus of claim 11, wherein the frame structure has a square shape when in an expanded position.

13. The pupil expansion apparatus of claim 12, wherein an angle formed at each corner of the square frame is 90 degrees when the pupil expansion apparatus is in its extended position.

14. The pupil expansion apparatus of claim 11, wherein the main beams can be folded to be in a collapsed position.

15. The pupil expansion apparatus of claim 1, further comprising:
one or more ring portions that are provided on the main beams.

16. The pupil expansion apparatus of claim 15, wherein two ring portions are provided at diagonally symmetrical positions each other in a plane formed by the frame structure.

17. The pupil expansion apparatus of claim 16, wherein a ring portion is provided on the main beam that has no ring portions.

18. The pupil expansion apparatus of claim 3, wherein the ends of the straight beams cross each other at their ends in the corners of the frame to form a cross beam arrangement, and the iris supporting portions are formed by one or more curved beams connected to the cross connected straight beams at the frame corner, which curved beams form an open curved shaped directed away from the frame to capture iris tissue.

19. The pupil expansion apparatus of claim 3, wherein the ends of the straight beams overlap each other at their ends in the corners of the frame with one above the other, the iris supporting portions are formed by curved beams connected to the overlapped ends of the straight beams at the frame corner, which curved beams form two generally parallel open curved shapes directed away from the frame to capture iris tissue, the two open curved shapes being connected together at their portions perpendicularly remote from the frame corners.

20. The pupil expansion apparatus of claim 3, wherein the main and sub beams of the frame are made of a single piece of wire.
